(19) United States
(12) Patent Application Publication
(10) Pub. No.: US 2007/0196927 A1
(43) Pub. Date: Aug. 23, 2007

(54) Method For Qualitative And/Or Quantitative Detection Of Polyethylene Glycols In Biological Fluids

(76) Inventors: Christian Grandfils, Jupille-sur-Meuse (BE); Guy Dandrifosse, Embourg (BE); Rene Remacle, Hannut (BE); Ibrahim Barakat, Woluwe-Saint-Pierre (BE); Patricia Deloyer, Angleur (BE)

Correspondence Address: KNOBBE MARTENS OLSON & BEAR LLP, 2040 MAIN STREET, FOURTEENTH FLOOR, IRVINE, CA, 92614, US

(21) Appl. No.: 10/570,784

(22) PCT Filed: Aug. 31, 2004
(86) PCT No.: PCT/EP04/51970

§ 371 Date: Oct. 2, 2006

(30) Foreign Application Priority Data
Sep. 4, 2003 (EP) 03077769.2
Mar. 18, 2004 (EP) 04075876.5

(51) Int. Cl. G01N 33/44

(52) U.S. Cl. 436/85

(57) ABSTRACT

The subject matter of the present invention relates a method for detection of polyethylene glycols (PEG) in an aqueous solution or in a sample of a biological fluid in a qualitative and/or semi-quantitative and/or quantitative manner, the method comprising the addition of a reagent containing iodine, alkali metal iodide and an acid; and a method for assessing the permeability of the intestine wherein two PEGs of different molecular weight, low molecular weight PEG (l.m.w. PEG) and high molecular weight PEG (h.m.w. PEG) are used as markers, wherein the l.m.w. PEG is able to cross the intestinal mucosa under physiological conditions and the h.m.w. PEG is essentially not able to cross the intestinal mucosa under physiological conditions, but is able to cross the intestinal mucosa under non-physiological, irregular conditions; and wherein h.m.w. and l.m.w. PEG concentrations are measured in a sample of urine from an individual after oral administration of h.m.w. PEG and l.m.w. PEG, wherein the differential index of h.m.w. PEG and l.m.w. PEG in the urine is calculated after determining the concentrations of h.m.w. PEG and l.m.w. PEG by using a reagent for quantitative detection of polyethylene glycol (PEG) in biological fluids, the reagent containing iodine, alkali metal iodide and an acid; and a kit for qualitative and/or semi-quantitative and/or quantitative detection of polyethylene glycol in biological fluids, e.g. in urine.

### FIELD OF THE INVENTION

The present invention relates to a method for qualitative and/or semi-quantitative detection and/or quantitative detection of polyethylene glycols in an aqueous solution or in biological fluids; a method for assessing the permeability biological barriers by detecting polyethylene glycols in biological fluids, especially a method for assessing the permeability of the intestine by detecting polyethylene glycols in biological fluids, and a kit.

The subject-matter encompasses a new method to measure the concentration of synthetic biocompatible macromolecules in biological fluids, particularly in order to evaluate the permeability of the intestine to macromolecules.

Polyethylene glycols are widely used as synthetic polymers in different fields, such as in the industry of food, agriculture, cosmetic, pharmaceutics and phytosanitary compounds as well as in medical applications.

The invention disclosed below is related to this class of polymers and the measurement of their concentrations in biological fluids.

For the present patent application, one of the main applications to measure polyethylene glycol concentration in biological fluids is the analysis of intestinal permeability, especially to macromolecules as explained below.

One essential function of the intestine is to work as a barrier in order to control the passage of molecules between intestinal lumen and blood compartments. Increase of this permeability does not only allow an anomalous passage of low molecular weight substances, but also, in certain circumstances, does permit antigens like proteins and viruses to cross the intestinal mucosa in high amounts. This passage may have pathological consequences like infections, inflammations, allergies and food intolerances. Up to now, commercially available permeability tests use only low molecular weight markers, such as lactulose, rhamnose, raffinose, cellobiose, or ⁵¹Cr-EDTA. However, these tests give only information on the intestinal permeability changes for low molecular weight products.

Polyethylene glycols (PEGs, chemical abstract n^{o} 25322-68-3) with a molecular weight in a range from 400 to 10,000 have also been reported as intestinal marker in the literature and some of them have been used for preliminary clinical tests. Polyethylene glycol (PEG) is defined as polyethylene glycol homopolymers having the generic formula H—[—O—CH₂—CH₂-]ₙ—OH or compounds containing one or several PEG sequences.

An increase of their intestinal permeability has been highlighted for example in Crohn disease and ulcerative colitis. However, the detection of these molecules in biological fluids like plasma or urine requires time-consuming analytical methodologies as well as expensive analytical instruments, limiting thereby their applications for routine analysis in clinics. Examples of these difficult analytical techniques are reported in the following references:
Eur. J. Clin. Chem. Clin. Biochem. 32,813-820 (1994)
J. Chromat. A800, 231-238 (1998)
J. Chromat. 565 (1-2), 297-307 (1991)
Analytical Letters, 20 (2), 293-301 (1987)
J. Chromat. B. 805,195-202, 2004

These techniques, which revealed to be difficult, time-consuming or expensive, were shortened by Hyden in 1955 [Hyden, S. The recovery of polyethylene glycol after passage through the digestive tract, Ann. R. Agric. Coll. Sweden 22, 411-424 (1955). As such designed, the method of Hyden requires to take a lot of precautions, due to the instability of the PEG-Ba²⁺-trichloroacetic acid emulsion. Malawer and Powell, [Malawer, S. J., Powell D. W.; An improved turbidimetric analysis of polyethylene glycol utilizing an emulsifier, Gastroenterology, 44, 250-256 (1967)], Buxton et al. [Buxton, T. B., Crockett, J. K., Morre, W. L., Moore, W. L., and Rissing J. P.; Protein precipitation by acetone for the analysis of polyethylene glycol in intestinal perfusion fluid, Gastroenterology, 76, 820-824 (1979)], Childs, [Childs, C. E., Microchemical J., 20, 190-192 (1975)], Ty [Ty, A. Microchemical J., 24, 287-290 (1979),], Skoog [Skoog, B., Vox Sang., 37, 345-349 (1979)], Sims and Snape [Sims G. E. C. and Snape, T. J., Anal. Biochem., 107, 60-63 (1980), ] and Bogdanova L. A. et al. (Document SU 19864026361 Database WPI, section Ch, Week 198837, Derwent Publications Ltd., London, GB; AN 1988-263362 XP002273163) have reported improvements of the assay, particularly by increasing its sensitivity. The methods of the state of art require the presence of barium or lithium to detect PEG. None of the methods of the state of art provide the possibility to differentially detect polyethylene glycols of different molecular weights and/or measure the concentration of polyethylene glycols of different molecular weights when polyethylene glycols of different molecular weights are present in the same sample.

The physico-chemical nature of the interaction between iodine and PEG has been detailed by others [Hemalatha, S. et al.; Complexation of molecular iodine by linear poly (ethylene glycol), Spectroscopy Letter, 12, 535-541 (1979)] and Szalbocs [Szalbocs, E., Über Iodkomplexe von hochpolymeren Polyethylenglycolen, Pharmazia, 39(4), 230-232 (1984)].

The object of the present invention was to provide a new, better and rapid method for detecting polyethylene glycol(s) and/or measuring the concentration of polyethylene glycol(s) (PEGs) in aqueous solutions or in biological fluids.

A further object of the present invention was to provide a method for detecting and/or measuring the concentration of a polyethylene glycol (PEG) of a specific molecular weight in aqueous solutions or in biological fluids, wherein in a given sample PEGs of different molecular weights are present.

A still further object of the present invention was to provide a rapid method for differentially detecting polyethylene glycols (PEGs) and/or measuring the concentration of polyethylene glycols (PEGS) in aqueous solutions or in biological fluids, wherein in a given sample high molecular weight (h.m.w.) and low molecular weight (l.m.w.) PEGs are present.

A further object of the present invention was to provide a method for assessing the permeability of biological barriers, particular of the intestine.

The object of the present invention was solved by a method for detection of polyethylene glycols (PEGs) in an aqueous solution or in a sample of a bioiogical fluid in a qualitative and/or semi-quantitative and/or quantitative manner, the method comprising the addition of a reagent containing iodine, alkali metal iodide and an acid.

In one preferred embodiment the polyethylene glycol solution, to be analysed, contains a low molecular weight polyethylene glycol (l.m.w. PEG). In this embodiment it is particularly preferred that the l.m.w. PEG used has a mean molecular weight below 1,000, more preferred a mean molecular weight in the range of 300 to 500, and most preferred a mean molecular weight of about 400. According to the present invention the l.m.w. PEG concentration can be measured also in the presence of h.m.w. PEGs. If a h.m.w. PEG is present in the sample, then the h.m.w. PEG preferably has a mean molecular weight in the range of 1,000 to 200,000, more preferred between 6,000 and 50,000 and most preferred between 6,000 and 25,000. As will be further outlined below in more detail, the present invention provides a method to detect and to measure the concentration of PEG of a specific molecular weight, in the presence of PEG having another specific molecular weight.

In an alternative preferred embodiment the polyethylene glycol solution to be analysed contains a high molecular weight polyethylene glycol (h.m.w. PEG). In this embodiment it is particularly preferred that the h.m.w. PEG used has a mean molecular weight in the range of 1,000 to 200,000, more preferred between 6,000 and 50,000 and most preferred between 6,000 and 25,000. According to the present invention the h.m.w. PEG concentration can be measured also in the presence of l.m.w. PEG. If a l.m.w. PEG is present, then the l.m.w. PEG has a mean molecular weight below 1,000, more preferred a mean molecular weight in the range of 300 to 500, and most preferred a mean molecular weight of about 400.

The present invention provides for the first time a method which can preferably be performed as semi-quantitative and/or quantitative measurement of the concentration of high molecular weight polyethylene glycol (h.m.w. PEG) and/or low molecular weight polyethylene glycol (l.m.w. PEG). Both h.m.w. PEG and l.m.w. PEG may even be present simultaneously. It is also possible to measure both h.m.w. PEG and l.m.w. PEG concentration in a given sample.

In a preferred embodiment the measurement of PEG concentration is performed under conditions where essentially no barium ions are present, preferably under conditions where essentially no barium ions and essentially no lithium ions are present.

In a further preferred embodiment of the present invention one molecular weight species of PEG is detected. The term “molecular weight species of PEG” means that the respective PEG has a specific molecular weight. For example, PEG 6,000 has a mean molecular weight of 6,000. However, the person skilled in the art will understand that not each molecule of PEG 6,000 will have exactly the same size. The term “molecular weight species of PEG” may also comprise PEG of a certain molecular weight range, for example 6,000 to 7,000; then the respective PEG has a mean molecular weight of 6,000 to 7,000. In that case the term “molecular weight range species of PEG” may be used.

Further, more preferred is that in the method the concentration of one molecular weight species of PEG is measured in the presence of another molecular weight species. The present inventors found conditions under which it is possible to measure the concentration of PEG of one molecular weight in the presence of further PEG(s) of different molecular weight(s), wherein these further PEG molecular weight species do not disturb the measurement of the first PEG species.

Further preferred the method is a method for differential detection of polyethylene glycols (PEGS) of different molecular weights or weight ranges. Preferably the concentration of polyethylene glycols (PEGs) of different molecular weights is differentially measured in an aqueous solution or in a sample of a biological fluid, wherein the concentrations of high molecular weight polyethylene glycol (h.m.w PEG) and of low molecular weight polyethylene glycol (l.m.w PEG) are separately measured in a sample.

Surprisingly, the present inventors have now found a method for qualitative and/or semi-quantitative and/or quantitative detection of polyethylene glycols (PEG) in aqueous solutions or in biological fluids by addition of a reagent containing iodine, alkali metal iodide and an acid and wherein barium or lithium ions are no more necessary to detect PEG by colorimetry. With this method according to the present invention it is possible to differentially detect polyethylene glycols of different molecular weights and/or to measure the concentration of polyethylene glycols of different molecular weights, i.e. the method of the present invention allows the distinct determination of two different size PEGs, when mixed in a sample, the one being of low molecular weight, the other of high molecular weight.

In a preferred embodiment of the method the biological fluid is roughly purified before the reagent is added. Preferably, the aqueous solution or the biological fluid is pre-treated by ion exchange resins or by adsorbant(s) before the reagent is added. This purification allows a more accurate measurement of PEG content, due to the removal of factors which might influence the reaction.

In a further preferred method the detection of polyethylene glycols is performed under conditions where essentially no barium or lithium ions are present in the reagent

If PEG is present in an aqueous solution or in a biological sample the mixing of the iodine reagent with the biological fluid induces a colour change of the solution. The optical density of the solution is related to the PEG concentration. Therefore, in a preferred embodiment of the method, after the addition of the reagent the presence of polyethylene glycol is determined by detecting a colour change. Preferably, this detection is performed by optical density measurement. For this measurement a wavelength between 400 and 700 nm is preferably used, most preferred a wavelength at 550 nm.

Polyethylene glycols (PEGS) to be detected by the method according to the present invention are defined as polyethylene glycol homopolymers having the generic formula:
H—[—O—CH₂—CH₂-]ₙ—OH
or compounds containing one or several PEG sequences. Preferably these compounds are covalently linked to one or several PEG sequences.

In particular, the reagent consists mainly of iodine, alkali metal iodide and an acid in an aqueous solution. The concentration of the acid is set between 10 mM and 5 M. As acid preferably acetic acid, boric acid or hydrochloric acid may be used, in particular acetic acid. As alkali metal iodide preferably potassium iodide may be used.

The method is suitable for determining polyethylene glycol concentrations, in a large range of molecular weights. But the method has also been adapted in order to determine the concentrations of both a low molecular weight PEG (l.m.w. PEG) and a high molecular weight PEG (h.m.w. PEG) present simultaneously in a biological fluid, i.e. an aqueous sample.

In particular the final concentration in the reaction mixture of iodine for the measurement of PEG concentration is adjusted between 0.1 and 20 mM, preferably between 1 and 15 mM.

The method according to the present application can preferably be used for the analysis of aqueous solutions or of biological fluids selected from the following group: urine, blood, blood plasma, interstitial fluid, lymph, cerebrospinal fluid and the like.

In more detail the method for differential detection of PEG in an aqueous solution or a sample of a biological fluid is performed as follows:
a) collecting a sample of an aqueous solution or a sample of a biological fluid comprising either one or both of high molecular weight polyethylene glycol (h.m.w. PEG) and low molecular weight polyethylene glycol (l.m.w. PEG);
b) for the determination of the h.m.w. PEG concentration:
   i) pretreatng the sample by ion exchange resins or by adsorbants;
   ii) adding a reagent containing iodine, alkali metal iodide and an acid to the pretreated sample,
      wherein the concentrations of iodine and the alkali metal iodide are adjusted in that way that essentially no colour reaction with the l.m.w. PEG but only with the h.m.w. PEG occurs;
   iii) determining optical density of the reaction mixture, eventually after a defined time;
c) for the determination of the l.m.w. PEG concentration:
   i) pretreating the sample by ion exchange resins or by adsorbants;
   ii) adding a reagent containing iodine, alkali metal iodide and an acid to the pretreated sample, wherein the concentration of iodine and the alkali metal iodide is adjusted in that way that the colour reaction occurs with the l.m.w. PEG;
   iii) determining optical density of the reaction mixture, eventually after a defined time.

In another embodiment of the method, the method further comprises after the pretreatment of the sample, a step for determining the h.m.w. or l.m.w. PEG concentration by using a calibration curve obtained with the pretreated aqueous solution or biological fluid. Preferably, the concentration of PEG is measured.

Advantageously such additional steps allow an immediate evaluation of an unknown amount of PEG added to the pretreated sample.

In a preferred embodiment of the method the concentrations of h.mw. PEG and l.m.w. PEG in the sample are calculated from the optical density measurements, and then the ratio of h.m.w. PEG concentration on l.m.w. PEG concentration is determined as differential index. When the measurement is performed with urine as biological liquid after ingestion of PEGs this differential index indicates the permeability of the intestine.

In a further preferred embodiment the method is a method for assessing the permeability of the intestine wherein two PEGs of different molecular weights, low molecular weight PEG (l.m.w. PEG) and high molecular weight PEG (h.m.w. PEG) are used as markers, wherein the l.m.w. PEG is able to cross the intestinal mucosa under physiological conditions and the h.m.w. PEG is essentially not able to cross the intestinal mucosa under physiological conditions, but is able to cross the intestinal mucosa under non-physiological, irregular conditions; and wherein h.m.w. and l.m.w. PEG concentrations are measured in a sample of urine from an individual after oral administration of h.m.w. PEG and l.m.w. PEG, wherein the differential index of h.m.w. PEG and l.m.w. PEG in the urine is calculated after determining the concentrations of h.m.w. PEG and l.m.w. PEG by using a reagent for quantitative or semi-quantitative detection of polyethylene glycol (PEG) in biological fluids, the reagent containing iodine, alkali metal iodide and an acid.

In a similar manner the permeability of other biological barriers can be measured. For example, the barrier between blood and brain: It is possible to inject PEGs of different but specific molecular weights into the blood vessel and to detect their appearance (or not appearance) in cerebrospinal fluid. Depending on the molecular weights one may be able to detect or not to detect PEG(s) in cerebrospinal fluid, and consequently one may define the permeability of the blood/brain barrier. Such measurements may apply also to other biological barriers. A further application is the determination, test or control of the permeability of artifical membranes, filters or filter systems as such, simply by detecting and measurement of which PEG of which molecular weight is passing and which is not passing.

In a preferred embodiment, the method for assessing the permeability of the intestine comprises the following steps:
a) collecting a sample of urine from an individual after oral administration of high molecular weight polyethylene glycol (h.m.w. PEG) and low molecular weight polyethylene glycol (l.m.w. PEG);
b) for the determination of the h m.w. PEG concentration:
   i) pretreating the urine sample by ion exchange resins or by adsorbants;
   ii) adding a reagent containing iodine, alkali metal iodide and an acid to the pretreated urine sample,
      wherein the concentrations of iodine and the alkali metal iodide are adjusted in that way that essentially no colour reaction with the l.m.w. PEG but only with the h.m.w. PEG occurs;
   iii) determining optical density of the reaction mixture, eventually after a defined time;
c) for the determination of the l.m.w. PEG concentration:
   i) pretreating the urine sample by ion exchange resins or by adsorbants;
   ii) adding a reagent containing iodine, alkali metal iodide and an acid to the pretreated urine sample, wherein the concentration of iodine and the alkali metal iodide is adjusted in that way that the colour reaction occurs with the l.m.w. PEG:
   iii) determining optical density of the reaction mixture, eventually after a defined time.

By pretreatment, one means treatment of urine to eliminate compounds that may interfere in colorimetric measurements. By ion exchange resins, one means cationic resin such as sulfonic acid resin, anionic resin such as quaterly ammonium resin or a mixture of them. By adsorbants, one means substances which interfere with or retain components of the biological fluid as for example charcoal.

For the determination of PEG a sample with known h.m.w. and/or l.m.w. PEG concentration is prepared and treated in the same way as the biological sample as reference.

In another embodiment of the method, the method further comprises after the pretreatment of the sample, a step for determining the concentration of h.m.w. or l.m.w. PEG by using a calibration curve obtained with the pretreated sample. Preferably, the concentration of PEG is measured.

Advantageously such additional steps allow an immediate evaluation of an unknown amount of PEG added to the pretreated sample.

In a preferred embodiment of the method the concentrations of h.mw. PEG and l.m.w. PEG in the sample are calculated from the optical density measurements, and then the ratio of h.m.w. PEG concentration on l.m.w. PEG concentration is determined as differential index. When the measurement is performed with urine as biological liquid after ingestion of PEGs this differential index indicates the permeability of the intestine.

The h.m.w. PEG and the l.m.w. PEG used in the biological sample, for example urine for receiving the calibration curve should have the same grade as the PEG administered to the patient.

In another embodiment of the method, the sample of step a) is divided in at least two portions before or after pre-treatment by ion exchange resins or by adsorbant(s) and the portions obtained are separately used in step b) and c).

The invention provides a method for the differential analysis of polyethylene glycols of different molecular weights in aqueous solutions or biological fluids. The inventive method is simple, sensitive, and rapid, allowing it to be carried out in a short time and using non toxic environmental friendly reagents. This method also provides an intestinal permeability diagnostic methodology, as PEGs can pass through the gastro-intestinal tract and the kidney barrier. The information acquired from this diagnostic tool is of importance for the identification and the following of several intestinal diseases or other disorders, actually known or unknown, but related to a modification of intestinal permeability.

The method also provides a simple tool to investigate the efficiency of galenic forms designed to improve the bio-availability of drugs, particularly those concerned with the administration of macromolecular drugs, such as peptides, proteins, nucleic acids, and the likes.

Taking into account that PEG could be used as biomarkers in the early phase of drug evaluation in exploratory development, especially in order to state precisely the therapeutic dose of a new drug, to establish a dose-response relationship and to define a plausible relationship between biomarker, pharmacological and pathogenesis, the determination of concentrations of PEGs of different molecular weights in biological fluids is also of high potential interest in the pharmaceutical industry.

The method according to the present invention is suitable for assessing the permeability of the intestinal barrier adopting two PEGs of different molecular weight as biomarkers. For example PEG of low molecular weight (PEG (l.m.w.)) can be used as simple passive diffusion biomarker reference, i.e. able to cross the intestinal mucosa without interacting with a plasma membrane component. On the other hand PEG of high molecular weight (PEG (h.m.w.)) can be used as abnormal diffusion biomarker, i.e. almost unable to cross the intestinal mucosa in physiological conditions.

In addition, the determination of the differential index of a h.m.w. PEG concentration on a l.m.w. PEG concentration in an urine sample allows to take into account of possible physiological variations such as gastro-intestinal transit time, diuresis and so on.

However, the method does not exclude to use the measurement of h.m.w. PEG concentration in the urine alone or by reference to (an)other substance(s) giving an information on physiological variations of gastro-intestinal properties.

In more details, the present invention is the description of an analytical technique which allows the measurement of PEG concentrations in biological fluids. The invention is particularly of interest because the assay as designed is rapid, sensitive, non expensive, specific and applicable to a wide range of molecular weight of PEGs (between 200 and 200,000, preferably between 400 and at least 25,000). Due to the selectivity of the assay, it can be performed on urine or any other biological liquid as previously defined. Due to the simplicity of the test and the non-toxicity of the reagents required to perform it, the assay could be performed for example by a patient himself, in order to get at least a semi-quantitative evaluation of his intestinal permeability by the time.

As such available, this new diagnostic tool could find applications in clinics, in particular in order to better understand the origin, development, follow-up of the following clinical disorders: infections (due to prions, viruses, bacteria), allergies, food intolerance, intestinal disorders or diseases (Crohn disease, celiac disease, gastro-enteritis). Also other inflammation processes which could occur in systemic organs or tissues as a consequence of the altered intestinal passage of antigens may be studied. Furthermore by taking into account the simplicity and low cost of the assay, it could also be used by people in order to evaluate possible intestinal permeability changes during some stresses such as sport activities.

Application of the PEG assay may, however, not be limited only to the intestinal permeability test as reported above. In particular, it is also well known from the literature that PEGs are extensively used as additives for different applications, i.e. food, drugs, cosmetic, ointments. It is thus also highly desirable to be able to monitor the concentration of these different PEGs in biological fluids, in particular because side effects have been reported in some toxicity studies [Lifton, L j., On the safety of “Golytely”, Gastroenterology, 86, 214-216 (1984); Sturgill, B. C., Herold, D. A., Bruns, D. E., Renal tubular necrosis in burn patients treated with topical polyethylene glycol, Lab Invest, 46, 81A (1982); Herold, D. A., Rodehaver, G. T., Bellamy, W. T. Fitton, L. A., Bruns, D. E., Edlich, R. F.; Tocicity of topical polyethylene glycol; Toxicol. Appl. Pharmcol, 65, 329-335 (1982)].

In addition, the PEGs are frequently used as non-absorbable markers to study water movements in human and animal transport studies (Jacobson, E. D., Bondy, D. C., Broitman, S. A. et al.; Validity of polyethylene glycol in estimating intestinal water volume, Gastroenterology, 44, 761-767 (1963).

There are also other properties of PEGs disclosed in the literature which justify the need to use the present invention to measure PEG concentration in biological specimen. In particular, PEGs have been used in order to concentrate by dialysis or to purify proteins using respectively their hydration and complexation properties.

For the method preferably the reagent for the determination of h.m.w. PEG concentration is used, which is prepared by the following steps:
a) iodine and alkali metal iodide are mixed under the solid state;
b) the mixture obtained in step a) is solubilized in water to obtain a final concentration of these two reactants iodine and alkali metal iodide of 50 mM and 230 mM, respectively;
c) use of the mixture of step b) as such, or
d) dilution of the mixture of step b) in an aqueous solution of alkali metal iodide by at least a factor of 2, preferably by at least a factor of 2.5, and most preferred by at least a factor of 2.78;
e) subsequent dilution in an acid.

The reagent for the determination of l.m.w. PEG is prepared by the following steps:
a) iodine and alkali metal iodide are mixed under the solid state;
b) the mixture obtained in step a) is solubilized in water to obtain a final concentration of these two reactants iodine and alkali metal iodide of 50 mM and 230 mM, respectively;
c) use of the mixture of step b) as such, or
d) dilution of the mixture of step b) in an aqueous solution of alkali metal iodide by at maximum a factor of 1.19;
e) subsequent dilution in an acid.

The concentration of the acid in the reagent used for method is between 10 mM and 5 M. Particularly, the acid may be selected from the following group: acetic acid, boric acid or hydrochloric acid, preferably acetic acid.

In order to measure the concentration of h.m.w. PEG without any interference of the presence of l.m.w. PEG the final concentration of iodine in the reaction mixture is adjusted to 0.1-5 mM, preferably to 1-5 mM, more preferred to 1-3.0 mM, even further preferred to 1.6-2.0 mM, more preferred to 1.8-2.0 mM, and most preferred to about 1.8 mM. For the measurement of l.m.w. PEG the final concentration of iodine in the reaction mixture is adjusted to higher than 5 mM, in particular it is adjusted to 5-20 mM, more preferred to 5-15 mM, even more preferred to 8-13 mM, and most preferred to 8.33-12.5 mM.

For the method the relation of the concentration of iodine: alkali metal iodide in the reagent is in the range of 1:1 to 1:10, preferably in the range of 1:2 to 1:7, more preferred 1:3 to 1:6, more preferred 1:4 to 1:5, and most preferred about 1:4,6.

Furthermore the alkali metal iodide used shall be preferably potassium iodide.

After the addition of the reagent the optical density is measured using a wavelength between 450 and 700 nm, preferably using a wavelength of 540 nm for PEG 400, and 480 nm for PEG 6,000. Particularly the optical density is measured at 550 nm.

The pretreatment of the PEG solution may be carried out by simple mixing of this solution with ion exchange resins. Such ions exchange resins may be anionic, cationic or a mixture of them.

When a mixture of cation exchange resin (CE) and anion exchange (AE) resin is used, the volume ratio urine:CE:AE is in the range 0.1:1:1 to 10:1:1; preferably in the range 0.4:1.1. to 5:1:1 and most preferred 1:1:1.

After mixture PEG solution and resins may be shaked during 2 to 50 minutes, preferably 15 minutes.

The h.m.w. PEG detected by the inventive method has a molecular weight in the range of 1,000 to 200,000, preferably between 6,000 and 50,000 and more preferred between 6,000 and 25,000.

The l.m.w. PEG used in this method has a mean molecular weight below 1,000, more preferred a mean molecular weight in the range of 300 to 500, and most preferred a mean molecular weight of about 400.

Using polyethylene glycols, especially a h.m.w and a l.m.w. PEG having these molecular weights, the status of the permeability of the intestine can be properly assessed.

It should be stressed that the l.m.w. PEG which is normally absorbed by a simple passive diffusion process into the intestinal mucosa, is used in the assessment of the intestinal permeability in order to avoid influences of gastro-intestinal transit time, diuresis and so on. Therefore the determination of the differential index of h.m.w. PEG concentration on l.m.w. PEG concentration in an urine sample allows to take into account of these possible physiological variations.

In order to give the best results in the measurement the final concentration of iodine in the reaction mixture may be varied depending on the exact molecular weight of the h.m.w. or l.m.w. PEG used in the assay. The iodine in the reaction mixture is adjusted to a final concentration of X [mM] depending on the molecular weight Y of the PEG according to the following table (table 1):

TABLE 1
The iodine in the reaction mixture is adjusted to a final concentration
of X [mM] depending on the molecular weight Y of the PEG.

| X | Y | |
|---|---|---|
| molecular weight of | Final concentration of | |
| PEG | iodine [mM] | |
| 200 ≦ X < 1000 | Y > 5 | |
| 200 ≦ X < 1000 | 5 < Y ≦ 20 | preferred |
| 200 ≦ X < 1000 | 5 < Y ≦ 15 | more preferred |
| 200 ≦ X < 1000 | 8 ≦ Y ≦ 13 | most preferred |
| 300-500 | Y > 5 | |
| 300-500 | 5 < Y ≦ 20 | preferred |
| 300-500 | 5 < Y ≦ 15 | more preferred |
| 300-500 | 8 ≦ V ≦ 13 | most preferred |
| 400 | Y > 5 | |
| 400 | 5 < Y ≦ 20 | preferred |
| 400 | 5 < Y ≦ 15 | more preferred |
| 400 | 8 ≦ Y ≦ 13 | most preferred |
| 1,000 ≦ X ≦ 200,000 | 0.1 ≦ Y ≦ 5 | |
| 1,000 ≦ X ≦ 200,000 | 1 ≦ Y ≦ 5 | preferred |
| 1,000 ≦ X ≦ 200,000 | 1 < Y ≦ 3 | more preferred |
| 1,000 ≦ X ≦ 200,000 | 1.8 < Y ≦ 2 | most preferred |
| 4,000 ≦ X ≦ 25,000 | 0.1 ≦ Y ≦ 5 | |
| 4,000 ≦ X ≦ 25,000 | 1 ≦ Y ≦ 5 | preferred |
| 4,000 ≦ X ≦ 25,000 | 1 ≦ Y ≦ 3 | more preferred |
| 4,000 ≦ X ≦ 25,000 | 1.8 < Y ≦ 2 | most preferred |
| 6,000 ≦ X ≦ 20,000 | 0.1 ≦ Y ≦ 5 | |
| 6,000 ≦ X ≦ 20,000 | 1 ≦ Y ≦ 5 | preferred |
| 6,000 ≦ X ≦ 20,000 | 1 ≦ Y ≦ 3 | more preferred |
| 6,000 ≦ X ≦ 20,000 | 1.8 < Y ≦ 2 | most preferred |

For the measurement of the permeability of the intestine, a l.m.w. PEG preferably with a molecular weight of about 400 and a h.m.w. PEG with a molecular weight from 4,000 to 25,000 will be chosen. For example, h.m.w. PEGs with 6,000, 10,000 and 20,000, respectively have been used in the examples described below. Generally, for the determination of l.m.w. PEG the final concentration of iodine will preferably be in the range of between 5 and 15 mM, more preferred between 8 and 13. For the determination of the h.m.w. PEG the final concentration of iodine in the reaction mixture is adjusted to 0.1-5 mM, preferably to 1-5 mM, more preferred to 1-3.0 mM, even more preferred to 1.8-2.0 mM, and most preferred to about 1.8 mM. The lower the molecular weight of the PEG is, the higher the final concentration of the iodine has to be used since less molecules of iodine will be bound to each molecule of PEG and vice versa.

However, if h.m.w. PEG of a lower range (for example 6,000 PEG) is used for the determination of the h.m.w. PEG, the person skilled in the art will choose a final concentration of iodine which is not too close to 5 mM in order to avoid a colour reaction also with PEG 400. In this case a lower final concentration of 1.8 mM may be used in order to adjust the iodine concentration in that way that essentially no colour reaction with PEG 400 will occur.

The object is also solved by a kit for qualitative and/or semi-quantitative and/or quantitative detection of polyethylene glycol in biological fluids containing:
a) a reagent for detecting high molecular weight polyethylene glycol without lithium and without barium ions, the reagent is further containing iodine, alkali metal iodide and an acid; and optionally
b) a reagent for detecting low molecular weight polyethylene glycol, without lithium and without barium ions, containing iodine, alkali metal iodide and an acid.

Preferably the kit is containing:
a) the reagent for detecting high molecular weight polyethylene glycol without lithium and without barium ions, the reagent is further containing 5 mM to 15 mM, preferably 9 mM iodine; 0.05 to 0.15 M alkali metal iodide, preferably 0.115 M alkali metal iodide; and 0.3 to 1 M of an acid, preferably 0.5 M of an acid; and optionally
b) the reagent for detecting low molecular weight polyethylene glycol, without lithium and without barium ions, the reagent is further containing 15 to 50 mM iodine, preferably 25 mM iodine; 0.05 to 0.15 M alkali metal iodide, preferably 0.115 M alkali metal iodide; and 0.3 to 1 M of an acid, preferably 0.5 M of an acid.

In a preferred embodiment the kit is further comprising:
c) high molecular weight polyethylene glycol, preferably PEG 6,000, preferably in a concentration of 0.5 to 5 mg/ml, most preferred 1 mg/ml.
d) low molecular weight polyethylene glycol, preferably PEG 400, preferably in a concentration of 30 to 100 mg/ml, most preferred 50 mg/ml.

In a further preferred embodiment the kit is containing ion exchange material or adsorbant(s). In a preferred embodiment the alkali metal is potassium iodide. In a further embodiment the acid is either acetic acid, boric acid or hydrochloric acid, preferably acetic acid.

In a further preferred embodiment the kit contains iodine, alkali metal iodide and an acid. In a preferred embodiment the acid is selected from the following group: acetic acid, boric acid or hydrochloric acid which may be contained in the kit, preferably acetic acid. As alkali metal iodide preferably potassium iodide is contained.

In a further preferred embodiment the kit contains PEG for use as positive control. In a particular preferred embodiment the kit contains a h.m.w. PEG and/or a l.m.w. PEG. The h.m.w. PEG has a mean molecular weight in the range of 1,000 to 200,000, preferably between 4,000 and 25,000 and more preferred between 6,000 and 20,000. The l.m.w. PEG used has a mean molecular weight below 1,000, more preferred a mean molecular weight in the range of 300 to 500, and most preferred a mean molecular weight of about 400.

### DESCRIPTION OF THE FIGURES

The present invention will be explained in the figures, which show the results of the examples described below.

FIG. 1 gives the optical densities of the reaction mixture obtained with three different urine samples containing different PEG 6,000 concentrations in presence of the reagent containing iodine, potassium iodide and acetic acid. The optical density was measured at 550 nm (n=3).

FIG. 2 gives the optical densities of the reaction mixture obtained with three different urine samples containing different PEG 20,000 concentrations in presence of the reagent containing iodine, potassium iodide and acetic acid. The optical density was measured at 550 nm (n=3).

FIG. 3 gives the optical densities of the reaction mixture obtained with three different urine samples containing different PEG 400 concentrations in presence of the reagent containing iodine, potassium iodide and acetic acid. The optical density was measured at 550 nm (n=3).

FIG. 4 shows the measurement of PEG 35,000 in water (♦) and three different urine samples (▴, ▪, ●), respectively, according to the methodology reported by Ty or Sims and Snape, but without purification step. The optical density was measured at 550 nm.

The following examples are given to illustrate the present invention. The scope of the invention, however, is not limited to the specific details of the examples.

### EXAMPLES

Intestinal Permeability Measurements

According to the present invention the most preferred way to conduct the diagnostic test of the intestinal permeability assessment, is performed in the following manner.

A given dose of the PEG(s), between 0.1 and 50 g, but preferably between 1 and 10 g, will be taken by the patient per os. The molecular weight of the PEG(s) could be in the range of 400 to 20,000. This PEG(s) could be taken either under the form of a solution, or under a dry form where PEG(s) is (are) incorporated in a galenic form, such as a gelatin capsule. The urine of the patient will be collected for 6 up to 24 hours after the PEG oral administration. Urine can be collected in any adequate bottle, provided that the recipient can be adequately closed and chemically cleaned. Without any restriction to other possibilities, one adequate material for the collecting bottle is polyethylene. During collection, urine is kept in a fridge, for example at 4° C. If PEG concentration measurement does not occur quickly after collection, collected urine can be conserved for example in a freezer, for example at −20° C. or with appropriate antibiotic(s). After thawing, urine sample may be centrifuged.

Next, the amount of PEG(s) recovered in urine is quantified in the following preferred way:

One volume of the collected urine is pretreated by one volume of a cation exchange resin (Amberlite 200 from Fluka) and one volume of an anion exchange resin (Amberlite IRA-900Cl from Supelco). The mixture of urine with resins is shaked for 15 minutes. Urine is then separated from resin by decantation and is called pretreated urine.

To a given volume of the pretreated urine a determined volume of an adequate reagent containing iodine and a given acid is added so that the final iodide concentration becomes 1.8 mM for h.m.w. and 12.5 mM for l.m.w. PEG.

The addition of this iodine reagent is performed in such a way to assure an intimate and rapid mixing with the biological sample. As so prepared, the combination of the sample with the iodine reagent is called the reaction mixture.

The iodine reagent is prepared according to the following steps:
iodine and potassium iodide are simultaneously solubilized in water to obtain a concentration of these two reactants of 50 mM and 230 mM respectively,
before the assay, this reagent is further diluted in an aqueous solution of potassium iodide. Dilution is carried out 2.78 times in potassium iodide 230 mM for the preparation of 9 mM potassium iodide used for h.m.w dosage. The resulting mixture is subsequently diluted 2 times more in acetic acid 1 M.

The mixing of the potassium iodine with PEG present in the biological sample induces a colour change of the solution whose optical density is related to the PEG concentration. The presence of a given PEG in urine can thus be visualized directly by the practitioner or eventually by the patient. But preferentially a precise quantification of PEG concentration in urine is desirable. A colorimetric analysis of the reaction mixture with reference to a calibration curve will allow to determine the PEG concentration in a range between 1 and at least 100 μg/ml of urine. In case of higher PEG concentration, a dilution of the urine sample is recommended.

The optical density is generally determined between 450 and 700 nm, preferentially at 550 nm.

### Example 1

### Colorimetric Calibration Curve of PEG 6,000 in Urine in the Presence of Fixed Concentrations of PEG 400

To a given volume of pretreated human urine are added PEG 400 and PEG 6,000 previously dissolved in water. The final concentrations of PEG 6,000 and PEG 400 in urine are ranged between 0 and 15 μg/ml and 50 and 200 μg/ml, respectively. To a given volume (0.8 ml) of this urine sample, 0.2 ml of an iodine reagent is added. The addition of this iodine reagent is performed in such a way to assure an intimate and rapid mixing with the biological sample. As so prepared, the combination of the sample with the iodine reagent is called the reaction mixture. The iodine reagent has been prepared according to the following steps:
iodine and potassium iodide, previously mixed under the solid state, have been simultaneously solubilized in water to obtain a final concentration of these two reactants of 50 mM and 230 mM respectively
this reagent is further diluted at least by 2.78 in an aqueous solution of potassium iodide and subsequently 2 times more in acetic acid. The final concentration of the acetic acid in the dilution has to be ranged preferentially between 10 mM and 5 M.
this iodine reagent can be stored during several months in a dark bottle before use.

For the determination of PEG 6,000 the final concentration of iodine in the reaction mixture in this example is 1.8 mM.

The mixing of the iodine reagent with PEG present in the biological sample induces a colour change of the solution with optical density is related to the polyether concentration. A colorimetric analysis of the reaction mixture allows to establish the calibration curve (eventually determined a given time after the mixing) which will allow to determine the PEG 6,000 concentration in a range between 1 and at least 30 μg/ml of urine. In case of higher PEG concentration, a dilution of the urine sample is recommended.

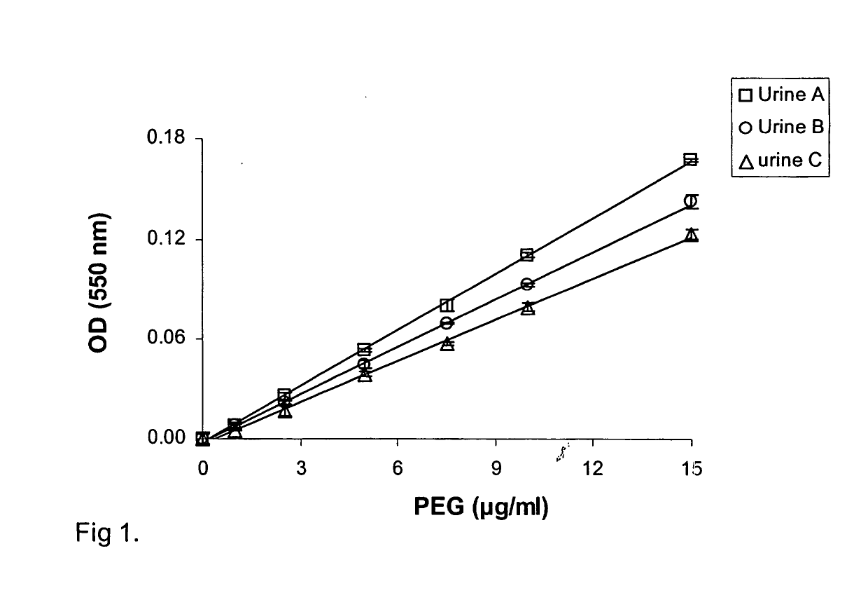

FIG. 1 corresponds to typical calibration curves for PEG 6,000 obtained in these specific conditions. It shows the measurement of PEG 6,000 concentration in three different urine samples. The optical density was measured at 550 nm.

The measurement of PEG is very simple and based on a change of colour of the reaction mixture, which can be detected visually. It can be estimated by reference to a colour scale calibration curve.

### Example 2

### Colorimetric Calibration Curve of PEG 20,000 in Urine

To a given volume of pretreated human urine is added PEG 20,000 previously dissolved in water. The final concentration of PEG 20.000 in urine is ranged between 0 and 15 μg/ml. To a given volume (0.8 ml) of this urine sample, 0.2 ml of the iodine reagent is added. The addition of this iodine reagent is performed in such a way to assure an intimate and rapid mixing with the biological sample. As so prepared, the combination of the sample with the iodine reagent is called the reaction mixture. The iodine reagent has been prepared according to example 1.

For the determination of PEG 20,000 the final concentration of iodine in the reaction mixture in this example is 1.8 mM.

The mixing of the iodine reagent with PEG present in the biological sample induces a colour change of the solution whose optical density is related to the polyether concentration. A colorimetric analysis of the reaction mixture allows to establish the calibration curve (eventually determined a given time after the mixing) which will allow to determine the PEG 20,000 concentration in a range between 1 and at least 30 μg/ml or urine. In case of a higher PEG concentration, a dilution of the urine sample is recommended.

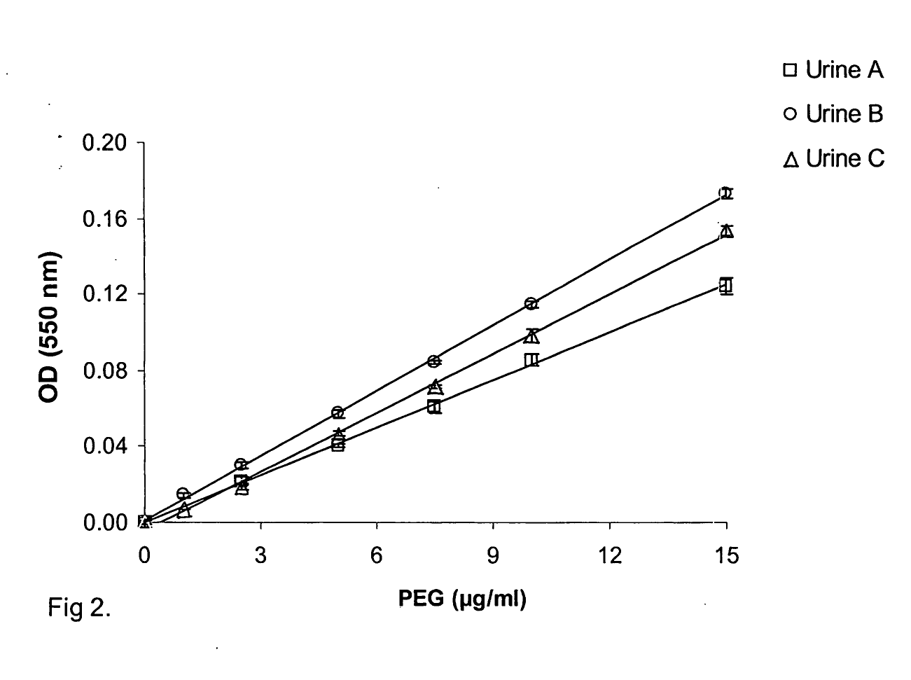

The optical density was measured at 550 nm. FIG. 2 corresponds to typical calibration curves obtained in these specific conditions. The figure shows the measurement of PEG 20,000 in three different urine samples.

### Example 3

### Colorimetric Calibration Curve of PEG 400 in Urine in the Presence of Fixed Concentrations of PEG 6,000.

To a given volume of pretreated human urine are added PEG 400 and PEG 6,000 previously dissolved in water. Final concentration of PEG 400 in urine is ranged between 0 and 140 μg/ml. To a given volume (0.8 ml) of this urine sample, iodine reagent is added (0.8 ml). The addition of this iodine reagent is performed in such a way to assure an intimate and rapid mixing with the biological sample. As so prepared, the combination of the sample with the iodine reagent is called the reaction mixture. The iodine reagent has been prepared according to the following steps:
iodine and potassium iodide, previously mixed under the solid state have been simultaneously solubilized in water to obtain a final concentration of these two reactants of 50 mM and 230 mM, respectively.
this reagent is used as such or further diluted by 1.19 in an aqueous solution of potassium iodide and subsequently 2 times more in acetic acid. The final concentration of the acetic acid in the dilution has to be ranged preferentially between 10 mM and 5 M.

this iodine reagent can be stored during several months in a dark bottle before use.

For the determination of PEG 400 the final concentration of iodine in the reaction mixture in this example is 12.5 mM.

The mixing of the iodine reagent with PEG present in the biological sample induces a colour change of the solution whose optical density is related to the PEG concentration. A colorimetric analysis of the reaction mixture allows to establish the calibration curve (eventually determined a given time after the mixing) which will allow to determine the PEG 400 concentration in a range between 20 and at 140 μg/ml or urine. In case of higher PEG concentration, a dilution of the urine sample is recommended.

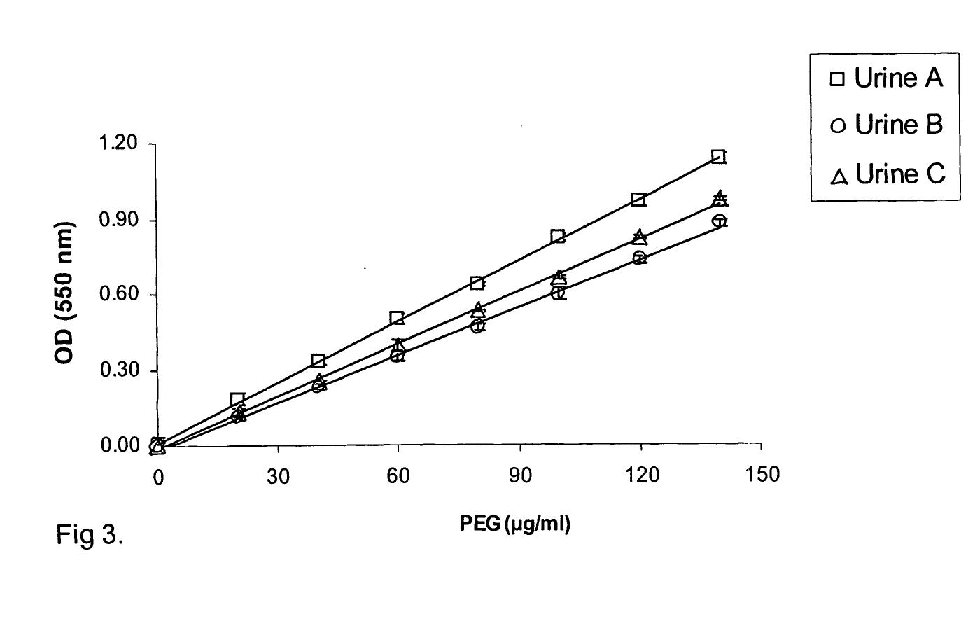

The optical density was measured at 550 nm. FIG. 3 corresponds to typical calibration curves obtained for PEG 400 in the presence of fixed concentrations of PEG 6,000.

### Example 4

### Counter-Example

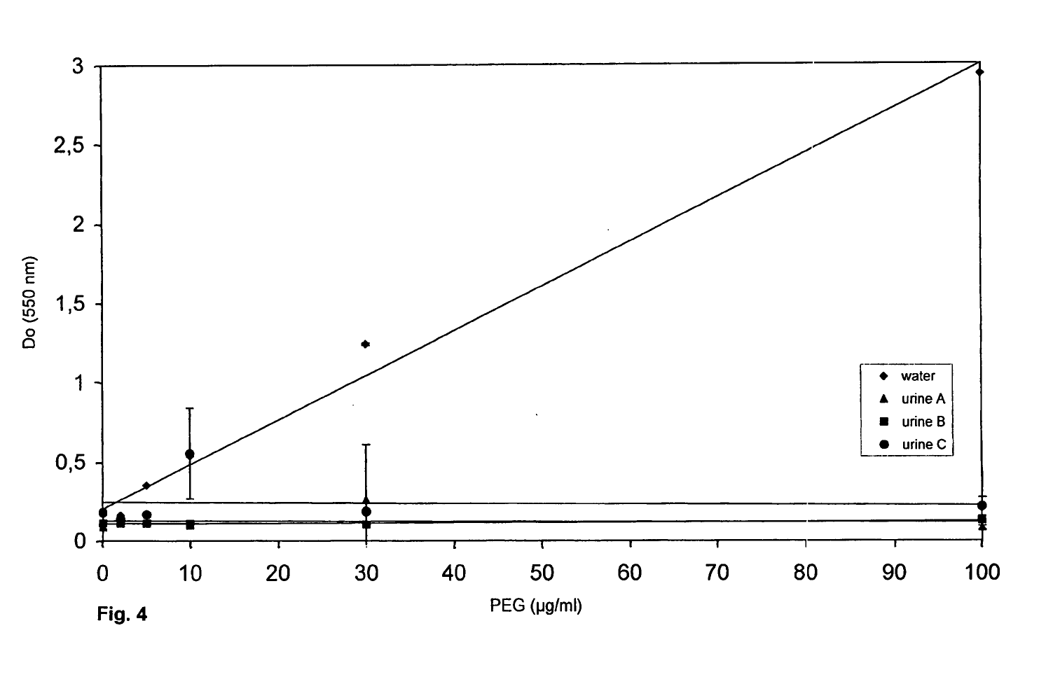

When the assay of PEG is performed according to the procedure reported by Ty or by Sims and Snape, but without any preliminary deproteinisation step, the presence of sulfate and phosphate ions in urine results in a precipitation of barium ions introduced by these authors to produce the colloid. As disclosed in FIG. 4, this precipitation of inorganic ions induces also the precipitation of PEG (in this example 35,000) (FIG. 4: curves ▴, ▪, ●) in the urine samples. No precipitation occurs in water (FIG. 4: curve ♦). This is explaining the absence of variation of the optical density in function of the PEG concentration when this latter is dissolved in the urine sample. The results are shown in FIG. 4. PEG 35,000 has been added to water (♦) and three different urine samples (▴, ▪, ●), respectively.

### Example 5

### Semi-Quantitative Evaluation of the Intestinal Permeability Measurement Carried Out by the Practitioner or Eventually by the Patient

The following example is given to explain the application of the ternary complex to analyze the intestinal permeability on a semi-quantitative basis by the practitioner or eventually by the patient.

According to the present invention, one of the most preferred ways to conduct the diagnostic test should be performed in the following manner:

A given dose of the low molecular weight PEG (between 0.1 and 50 g, but preferably between 1 and 10 g of PEG 200-1000) and a high molecular weight PEG (between 0.1 and 50 g, but preferably between 1 and 10 g of PEG 6,000-20,000), could be taken by the patient either under the form of a solution or under a dry form where PEGs are incorporated in a galenic form, such as a gelatin capsule. The urine will be collected by the patient for 6 up to 24 hours after the PEG oral administration. During collection, urine is kept in a fridge, for example at 4° C. The urine will be submitted to a pretreatment, and mixed with a determined volume of an iodine reagent. The addition of this reagent is performed in such a way to assure an intimate and rapid mixing with the biological sample. In this case, the iodine reagent, made commercially available, will have the composition given in example 1. According to the range of molecular weight of PEG taken orally by the patient a significant modification of the colour intensity of the mixture appearing just after mixing will attest an alteration in intestinal permeability. The extent of colour intensity variation will be appreciated by the practitoner or eventually the patient with the help of a given reference colour scale providing a semi-quantitative evaluation of the intestinal permeability modification.

### Example 6

### Quantitative Evaluation of the Intestinal Permeability Measurement Carried Out in a Clinical Laboratory

The following example is given to explain the application of PEGs to analyze on a (semi)-quantitative basis the intestinal permeability using colorimetric analysis which can be applied for example in a clinical laboratory. According to the present invention a preferred way to conduct the diagnostic test should be performed in the following manner:

A given dose of the low molecular weight PEG (between 0.1 and 50 g, but preferably between 1 and 10 g of PEG 200-1000) and a high molecular weight PEG (between 0.1 and 50 g, but preferably between 1 and 10 g of PEG 6,000-20,000), could be taken by the patient either under the form of a solution or under a dry form where PEGs are incorporated in a galenic form, such as a gelatin capsule. The urine will be collected by the patient for 6 up to 24 hours after the PEG oral administration. During collection, urine is kept in a fridge, for example at 4° C. If PEG concentration measurement does not occur immediately after collection, collected urine is conserved in a freezer, for example at −20° C. or with appropriate antibiotic(s). After thawing, urine sample may be centrifuged. The amount of both high and low molecular weight PEG recovered in urine could be quantified following the sequential steps described below:
step 1: quantification of the high molecular weight PEG:

To a given volume (0.8 ml) of a pretreated urine sample, 0.2 ml of iodine reagent is added. The addition of this reagent is performed in such a way to assure an intimate and rapid mixing with the biological sample. As so prepared, the combination of the sample with the iodine reagent is called the reaction mixture. The iodine reagent has been prepared according to the composition given in example 1.

The mixing of the iodine reagent with PEG present in the biological sample induces a colour change of the solution whose optical density is related to the PEG concentration. A colorimetric analysis of the reaction mixture with reference to a calibration curve (eventually prepared by using urine and determined a given time after the mixing) will allow to determine the PEG concentration in a range between 1 and at least 30 μg/ml of urine. In case of higher PEG concentration, a dilution of the urine sample is recommended.

The optical density is generally determined between 450 and 700 nm, preferentially at 550 nm. In this specific concentration of iodine present in the reagent mixture, the presence of l.m.w. PEG 400 in the urine sample does not significantly interfere with analysis of the high molecular weight PEG.
step 2: quantification of l.m.w. PEG:

Once the h.m.w. PEG concentration is known, its absorption contribution can be subtracted from the analysis of l.m.w. PEG.

To a given volume (0.8 ml) of this urine sample, iodine reagent (0.8 ml) is added. The addition of this iodine reagent is performed in such a way to assure an intimate and rapid mixing with the biological sample. As so prepared, the combination of the sample with the iodine reagent is called the reaction mixture. The iodine reagent has been prepared according to example 3.

The mixing of the iodine reagent with PEG present in the biological sample induces a colour change of the solution whose optical density is related to the PEG concentration. A colorimetric analysis of the reaction mixture allows to establish the calibration curve (eventually prepared by using urine and determined a given time after the mixing) which will allow to determine the PEG 400 concentration in a range between 25 and 150 μg/ml of urine. In case of higher PEG concentration, a dilution of the urine sample is recommended. The optical density is generally determined between 450 and 700 nm, preferentially at 550 nm.

### Example 7

### Quantitative Evaluation of the Intestinal Permeability Measurement Carried Out in a Clinical Laboratory, Using Microtiterplate Spectrophotometer

The following example is given to explain the application of the ternary complex to analyze on a (semi)-quantitative basis the intestinal permeability using colorimetric analysis which can be applied for example in a clinical laboratory. According to the present invention a preferred way to conduct the diagnostic test should be performed in the following manner:

2 g of PEG 400 and 10 g of PEG 6,000 are taken by a patient either under the form of a solution or under a dry form where PEGs are incorporated in a galenic form, such as a gelatin capsule. Urine is then collected by the patient for 8 hours after PEG oral administration. During collection, urine is kept in a fridge at 4° C. If PEG concentration measurement does not occur immediately (for example 48 h) after collection, the collected urine is conserved in a freezer, for example at −20° C. or with appropriate antibiotic(s). After thawing, urine sample may be centrifuged. The amount of both high and low molecular weight PEG recovered in urine could be quantified following the sequential steps described below:
step 1: urine sample pretreatment:

To 1 ml of urine is added 1 ml of a cation exchange resin (Amberlite 200 from Fluka) and 1 ml of an anion exchange resin (IRA-900Cl from Supelco). The mixture of urine with resins is shaked for 15 minutes. Urine is then separated from resin and is called pretreated urine.
step 2: quantification of the high molecular weight PEG, PEG 6,000:

To a given volume (100 μl) of the pretreated urine sample, 25 μl of iodine reagent are added. The addition of this reagent is performed in such a way as to assure an intimate and rapid mixing with the pretreated urine sample. As so prepared, combination of pretreated urine sample with iodine reagent is called reaction mixture. The iodine reagent has been prepared according to the composition given in example 1.

The mixing of the iodine reagent with PEG present in the pretreated urine sample induces a colour change of the solution whose optical density is related to the PEG concentration. A colorimetric analysis of the reaction mixture with reference to the same urine sample added of a known concentration of PEG 6,000 (between 5 and 40 μg/ml, preferentially 20 μg/ml) and pretreated in the same way will allow to determine the PEG 6,000 concentration. The optical density is determined at 480 nm.
step 3: quantification of l.m.w. PEG, PEG 400:

To a given volume (100 μl) of the pretreated urine sample, iodine reagent (100 μl) is added. The addition of this iodine reagent is performed in such a way as to assure an intimate and rapid mixing with the pretreated urine sample. As so prepared, the combination of the urine sample with the iodine reagent is called the second reaction mixture. The iodine reagent has been prepared according to example 3.

Related to the dose of PEG 400 ingested (2 g) and to metabolic characteristics of each patient, a dilution of the urine sample is applied, generally between 2 up to 30 times.

The mixing of the iodine reagent with PEG present in the urine pretreated sample induces a colour change of the solution whose optical density is related to the PEG concentration. A colorimetric analysis of the second reaction mixture with reference to the same urine sample added of a known concentration of PEG 400 (from 200 μg/ml up to 3 mg/ml depending on the dilution factor to be applied) and pretreated in the same way will allow to determine the PEG 400 concentration. The optical density is determined at 540 nm.

### Example 8

### TPI 6000 Kit for Colorimetric Detection of Polyethylene Glycols in Urine.

A colorimetric detection kit is now ready to use and corresponds to the method of detection of polyethylene glycol illustrated in example 7. A protocol of use has been prepared and is reported hereafter.

Measurement of Intestinal Permeability to Macromolecules by Colorimetric Determination of Urinary PEG.

P-EL-24

1. Clinical Application

All pathologies for which a change of the intestinal permeabillity to macromolecules is suspected: Crohn disease, ulcerus rectocolitis, rheumatoid polyarthritis, allergies, food intolerance.

2. Principle of the Assay

The measure of intestinal permeability to macromolecules is based on the detection in urine of two polyethylene glycols orally administrated. One, PEG 400, of low molecular weight is used as reference. The other, PEG 6,000, with a molecular weight representative of the macromolecules. The determination of these materials is realized by colorimetry after preparing the samples on ion-exchange medium and adding adequate reactive. Intestinal permeability index is expressed as the ratio between the amount of excreted PEG 6,000 and the amount of excreted PEG 400.

In a first stage, the amount of PEG 6,000 in urine is measured.

In a second stage, the amount of PEG 400 in urine is measured.

The Intestinal Permeability Index is calculated as:
IPI=(PEG 6,000exc/PEG 400exc)×100

Because there is a great difference between the composition of the urine, it is necessary to perform a dilution test to determine the amount of PEG 400 that will be added.

3. Material Provided

The TPI 6000 kit contains the reagents needed to carry out 24 determinations.

All the reagents must be kept at room temperature.

Expiration dates of each reagent are indicated on its labels.

1. PEG 6,000: 1 vial (1 mL) polyethylene glycol 6,000. Ready for use.

2. PEG 400: 1 vial (2 mL) polyethylene glycol 400. Ready for use.

3. I₂/KI solution A: 2 vials (1 mL) I₂/KI solution. Ready for use. After opening of the vial, the solution can be used for 5 days.

4. I₂/KI solution B: 2 vials (8 mL) I₂/KI solution. Ready for use. After opening of the vial, the solution can be used for 5 days.

5. Tubes: 3×24 tubes containing a mixture of ion exchange beads. Ready for use.

6. Microtiterplates: 2×1 microtiterplate. Ready for use.

4. Kit Reagents

TABLE 2
The following reagents may be provided in the kit.

| Reagents | Quantity | Physical state | Concentrations |
|---|---|---|---|
| PEG 6,000 | 1 × 1 mL | Ready for use | PEG 6,000: 1 mg/ml |
| PEG 400 | 1 × 2 mL | Ready for use | PEG 400: 50 mg/ml |
| I₂/KI A | 2 × 1 mL | Ready for use | iodine: 9 mM |
| | | | KI: 0.155 M |
| | | | acid: 0.5 M |
| I₂/KI B | 2 × 8 mL | Ready for use | iodine: 25 mM |
| | | | KI: 0.155 M |
| | | | acid: 0.5 M |
| Sorb EX | 3 × 24 tubes | Ready for use | |
| Microtiterplate | 2 × 1 | Ready for use | |

5. Materials Required but not Supplied

Adjustable automatic pipettes with disposable tips.

Polyethylene Pasteur pipettes

Distilled water.

Roller shaker.

Spectrophotometer for microtiterplates able to read at 480 and 540 nm.

6. Warnings and Precautions

For in vitro diagnostic use only.

In order to avoid personal and environmental contamination, the following precautions must be observed:

Use disposable gloves while handling potentially infectious material and performing the assay.

Do not pipette reagents by mouth.

Do not smoke, eat, drink or apply cosmetics during the assay.

Avoid contact with skin, eyes and mucous membranes. In case of accident wash thoroughly with soap and rinse with water.

All samples and reagents used for the assay must be considered potentially infectious; therefore, the assay waste must be decontaminated and disposed off in accordance with established safety procedures. Disposable ignitable material must be incinerated; disposable non-ignitable material must be sterilized in autoclave for at least 1 hour at 121° C.

Elimination of iodine solutions must be done in accordance with local regulations; note: colorations due to iodine can be eliminated by use of sodium thiosulphate

In order to obtain reproducible results, the following rules must be observed:

Do not mix reagents from different lot numbers or from other manufacturers;

Do not freeze reagents.

Strict adherence to the specific time and temperature of incubations is recommended for accurate results.

Do not use reagents beyond their expiry date.

Use thoroughly clean glassware.

Use distilled water, stored in clean containers.

Avoid any contamination among samples; for this purpose, disposable tips should be used for each sample and reagent.

Cross contaminations of reagents or sample could cause false results. Use a clean, fresh, disposable pipette tip for each reagent or specimen manipulation.

Do not expose the reactives containing iodine to air. Close hermeticaly the vial after use.

Follow exact incubation times.

Before using, mix well each urine sample.

A variety of factors influence the assay performance. These include the accuracy

and reproducibility of pipetting technique, the photometer used, timing bias during the assay.

7. Specimen Storage

Urine samples can be stored at 2-8° C. for maximum two days.

For longer storage, freeze the samples at −20° C. for maximum two weeks.

Repeated freezing and thawing of samples should be avoided.

8. Assay Procedure

1. Preparation of Exchange Medium

A. Hydration of the Exchange Medium:

Prepare 3 tubes of exchange medium per urine sample.

Hydration of exchange medium is preferably realized the day before the assay but never more than 4 days before.

1. Mix the tubes to obtain an homogenous mixture of the two components.

2. Open delicately the tubes and add 3.9 mL of distilled water.

3. Close the tubes and agitate well to put all the exchange medium in suspension.

4. Place the tubes on a rack, control that all the exchange medium is submerged in water. If necessary, delicately tap the tubes on the laboratory bench.

5. Store the hydrated exchange medium at 4° C.

B. Washing of the Exchange Medium:

Washing of the exchange medium can be performed the day before or the day of the assay.

1. Open delicately the tubes of hydrated exchange medium.

2. Aspirate gently the excess of water up to the top of the exchange medium, avoid to remove exchange medium beads. Use a polyethylene Pasteur pipette. For a better aspiration of the washing water, tamp the exchange medium down by delicately tap the tubes on the laboratory bench.

3. Add 2 mL of distilled water, close the tubes and mix well.

4. Repeat twice the operations 1 to 3.

5. Store the washed exchange medium at 4° C. excepted if the washing procedure has been made the day of the assay.

2. Assay:

Bring washed exchange medium and urine samples at room temperature.

1. Centrifuge 4-10 mL of each urine sample at 700 g for 15 min. Note the volume of collected urine in 8 hours.

2. Open delicately the tubes of washed exchange medium. Aspirate gently the excess of water up to the top of the exchange medium, avoid to remove beads. Use a polyethylene Pasteur pipette. Terminate this operation by using an automatic pipet. Delicately tap the tubes on the laboratory bench so that all the exchange medium are pratically at the same level.

A. PEG 6,000 Assay

1. For each urine sample, prepare 2 exchange medium tubes, noted ‘0’ and ‘6000 ’

2. In the ‘0’ tube, add 1 mL of centrifugated urine sample.

3. In the ‘6000’ tube, add 1 mL centrifugated urine sample and 20 μL of PEG 6,000 solution.

4. Close the tubes and immediately incubate for 15 min. at room temperature on a roller shaker. Be sure that the exchange medium and urine sample mixture is homogenous.

5. After incubation, collect the supernatant of the treated urine samples and note them ‘0-R’ and ‘6000-R’. Let rest for 2 hours at room temperature.

6. Add 100 μL of each supernatant in the wells of a microtiterplate.

7. Add 25 μL of I2/KI-A solution.

8. After 15 min incubation at room temperature and protected from light, read the absorbance at 480 nm.

B. PEG 400 Dilution Test

Because there is a great difference between the composition of the urine, it is necessary to perform a dilution test to determine the amount of PEG 400 that will be added.

1. Take the supernatants noted ‘0’ and dilute them 4, 8, 16 and 32-fold with distilled water.

2. Add 100 μL of each dilution in the wells of a microtiterplate.

3. Add 100 μL of I2/KI-B solution.

4. After 15 min incubation at room temperature and protected from light, read the absorbance at 540 nm.

C. PEG 400 Assay

1. For each urine sample, prepare 1 exchange medium tube, noted ‘400’.

2. In the ‘400’ tube, add 1 mL of centrifugated urine sample and X μL of PEG 400 solution. For the determination of the volume X to be added, the table 3 should be consulted.

TABLE 3
Determination of the volume X to be added for the
measurement of PEG 400.

| Dilution | Absorbance at | Dilution to be | | |
|---|---|---|---|---|
| test | 540 nm | applied | X (μl) | Y (μg/ml) |
| 4x | <0.90 | 2x | 4 | 200 |
| | 0.90-1 | 4x | 8 | 400 |
| 8x | 0.95 | 6x | 12 | 600 |
| | 0.95-1.05 | 8x | 16 | 800 |
| | >1.05 | 10x | 20 | 1000 |
| 16x | <0.95 | 12x | 24 | 1200 |
| | 0.95-1.05 | 16x | 32 | 1600 |
| | >1.05 | 20x | 40 | 2000 |
| 32x | <0.95 | 25x | 50 | 2500 |
| | 0.95-1.05 | 30x | 60 | 3000 |

3. Close the tubes and immediately incubate for 15 min. at room temperature on a roller shaker. Be sure that the exchange medium and urine sample mixture is homogenous.

4. After incubation, collect the supernatant of the treated urine samples and note them ‘400-R’. Let rest for 2 hours at room temperature.

5. Apply dilution to each supernatant ‘0-R’ and ‘400-R’.

6. Add 100 μL of each diluted supernatant ‘0-R’ and ‘400-R’ in the wells of a microtiterplate.

7. Add 100 μL of I2/KI-B solution.

8. After 15 min incubation at room temperature and protected from light, read the absorbance at 540 nm.

9. Calculation and Interpretation of the Results

Amount of excreted PEG 6000:
PEG 6000_{exc}(mg)=[(A₀-0.300)×20/(A₆₀₀₀-A₀)]×V

A₀=absorbance at 480 nm for ‘0-R’ solution

A₆₀₀₀=absorbance at 480 nm for ‘6000-R’ solution

V=volume of collected urine (8 h)

Amount of excreted PEG 400:
PEG 400_{exc}(mg)=[(A₀-0.810)×Y/(A₄₀₀-A₀)]×V

where A₀=absorbance at 540 nm for ‘0-R’ solution

A₄₀₀32 absorbance at 540 nm for ‘400-R’ solution

Y=amount of PEG 400 added with X μL

V=volume of collected urine (8 h)

Intestinal Permeability Index: IPI=(PEG 6,000_{exc}/PEG 400_{exc})×1001

IPI normal value: 0- 0.135%

10. Performances of the Assay

Precision

Precision was evaluated upon intra- and inter-assay variability.

TABLE 4
Precision of meaurement of PEG 6,000.

| | | Mean | Standard | CV |
|---|---|---|---|---|
| Samples | n | (μg/mL) | deviation | (%) |
| 1 | 8 | 1.61 | 0.53 | 32.7 |
| 2 | 8 | 5.7 | 0.81 | 14.2 |
| 3 | 8 | 14.6 | 1.59 | 10.9 |

Intra-Assay (PEG 400)

TABLE 5
Precision of meaurement of PEG 400.

| | | Mean | Standard | CV |
|---|---|---|---|---|
| Samples | n | (μg/mL) | deviation | (%) |
| 1 | 8 | 462 | 43 | 9.3 |
| 2 | 8 | 866 | 101 | 11.7 |
| 3 | 8 | 1770 | 69 | 3.9 |

Inter-Assay

TABLE 6
Precision of meaurement of PEG 6,000 and PEG 400.

| | | Mean | Standard | CV |
|---|---|---|---|---|
| Samples | n | (μg/mL) | deviation | (%) |
| PEG 6,000 | 4 | 2.17 | 0.38 | 17.6 |
| PEG 400 | 4 | 787 | 65 | 8.3 |

Sensitivity

The analytical sensitivity is expressed as the minimal absorbance value significantly different from the absorbance obtained for a sample that contain no PEG.

This absorbance value is 0.335 for the PEG 6,000 measurement and 0.875 for the PEG 400 measurement.

11. Scheme of the Assay

1. PEG 6,000

TABLE 7
Scheme of the assay of PEG 6,000

| Hydration exchange medium | |
|---|---|
| Washing of the exchange medium | |
| Centrifugation of urine samples | |
| TUBE ‘6000’ | TUBE ‘0’ |
| 1 mL centrifugated | 1 mL centrifugated urine |
| urine | |
| 20 μL PEG 6,000 | |
| Add to exchange medium | |
| Incubate 15′ on a shaker at RT | |
| Supernatant ‘6000-R’ | Supernatant ‘0-R’ |

2 h rest at room temperature

| | |
|---|---|
| 100 μL supernatant in well | 100 μL supernatant in well |
| 25 μL I₂/KI-A | 25 μL I₂/KI-A |

Incubate 15′ at RT
read at 480 nm
Note absorbances for further IPI calculation

2. PEG 400

TABLE 8
Scheme of the assay of PEG 400

| TUBE ‘0’ | | | | TUBE ‘400’ |
|---|---|---|---|---|
| Supernatant ‘0-R’ | | | | |
| Dilution 4, 8, 16, 32 fold with distilled water | | | | |
| DIL4 | DIL8 | DIL16 | DIL32 | |
| 100 | 100 | 100 | 100 | |
| μL/well | μL/well | μL/well | μL/well | |

| | |
|---|---|
| 100 μL I₂/KI-B | |
| Incubate 15′ at RT | |
| read at 540 nm | |
| Determine the volume X of PEG 400 to be | |
| added in tube ‘400’ | |
| | 1 mL centrifugated urine |
| | X μL PEG 400 |
| | Add to exchange medium |
| | Incubate 15′ on a shaker at |
| | RT |
| | Supernatant ‘400-R’ |
| | 2 h rest at room |
| | temperature |

Dilute the supernatants

| | |
|---|---|
| 100 μL diluted | 100 μL diluted supernatant ‘400-R’ |
| supernatant ‘0-R’ | |
| 100 μL of I₂/KI-B | 100 μL of I₂/KI-B |

Incubate 15′ at RT, protect from light
read at 540 nm
Note absorbances for further IPI calculation

### Example 9

### Estimation of the Intestinal Permeability of Healthy Volunteers

The kit of example 8 was used to estimate the intestinal permeability of healthy volunteers under conditions as summarized in table 9. The volunteers ingested the PEGS as described in Example 6. The “drug” was given to the volunteers once about 20 h before the ingestion of PEGs following the instructions given in the notice accompanied the box in which the “drug” is provided. The oral administrations of PEG were realised at an interval of at least one week. The references refer to the following drugs: (a) Reparil®, (b) Body Sculpt®, (c) Venoplant®, (d) Chitosan®. The test was applied to urine maintained at 4° C. for about 20 h. As shown in Table 9, the normal values for IPI (Intestinal Permeability Index) lie within the range of between 0 and 0.135 %. In the experimental conditions used, Body Sculpt® increases the IPI above its normal value (0-0.135%) or maybe decreases the intestinal permeability of macromolecules in vivo. This observation suggests that Body Sculpts® has the capacity to modify the intestinal permeability to macromolecules. An increase followed by a decrease of permeability (or the reverse) could be due to a fine adjustment of the opening degree of the “tight junctions” by the drug.

TABLE 9
IPI values (%) of volunteers without apparent disease by using the
TPI 6000 kit. Effect of “drugs” containing, among others, a substance
able to increase the intestinal permeability in vitro.

| Volunteers | | | | |
|---|---|---|---|---|
| (#) | Without drug | With drug | Without drug | With drug |
| 2 | 0.091 | 0.082 (a) | 0.065 | 0.075 (d) |
| 1 | 0.104 | — | 0.037 | 0.044 (d) |
| 8 | 0.047 | 0.012 (b) | — | — |
| 9 | 0.072 | 0.018 (b) | — | — |
| 3 | 0.096 | 0.195 (b) | 0.134 | 0.081 (b) |
| 7 | 0.051 | 0.232 (b) | — | — |
| 4 | 0.116 | 0.033 (c) | — | — |
| 6 | 0.064 | 0.131 (c) | — | 0.180 (b) |
| 5 | 0.084 | 0.112 (d) | 0.006 | 0.032 (c) |

1. A method for detection of polyethylene glycols (PEGS) in an aqueous solution or in a sample of a biological fluid in a qualitative and/or semi-quantitative and/or quantitative manner, the method comprising separately measuring concentrations of high molecular weigh polyethylene glycol (hmw PEG) and low molecular weight polyethylene glycol (lmw PEG) in said aqueous solution or in said sample of a biological fluid.

2. The method according to claim 1, wherein the measuring of PEG is performed under conditions where essentially no barium ions are present.

3. (canceled)

4. (canceled)

5. The method of claim 1, wherein the method is a method for differential detection of polyethylene glycols (PEG) of different molecular weights, wherein the one molecular weight species of PEG is measured in the presence of the other molecular weight species of PEG.

6. (canceled)

7. (canceled)

8. The method of claim 1, wherein after the addition of the reagent the presence of polyethylene glycol is determined by detecting a color change.

9. (canceled)

10. The method according to claim 1, wherein both hmw PEG and lmw PEG are present simultaneously in the aqueous solution or in the biological fluid to be measured.

11. (canceled)

12. The method according to claim 1, wherein aqueous solutions are analyzed or biological fluids selected from the group: urine, blood, blood plasma, interstitial fluid, lymph, cerebrospinal fluid and the like.

13. The method of claim 1, for assessing the permeability of the intestine wherein two PEGs of different molecular weights, low molecular weight PEG (lmw PEG) and high molecular weight PEG (hmw PEG) are used as markers, wherein the lmw PEG is able to cross the intestinal mucosa under physiological conditions and the hmw PEG is essentially not able to cross the intestinal mucosa under physiological conditions, but is able to cross the intestinal mucosa under non-physiological, irregular conditions; and wherein hmw and lmw PEG contents or concentrations are measured in a sample of urine from an individual after oral administration of hmw PEG and lmw PEG, wherein the differential index of hmw PEG and lmw PEG in the urine is calculated after determining the concentrations of hmw PEG and lmw PEG by using a reagent for quantitative detection or semi-quantitative detection of polyethylene glycol in biological fluids.

14. The method of claim 13, comprising the steps of:
a) collecting a sample of an aqueous solution or a sample of a biological fluid comprising either one or both of high molecular weight polyethylene glycol (hmw PEG) and low molecular weight polyethylene glycol (lmw PEG);
b) for the determination of the hmw PEG concentration:
i) pretreating the sample by ion exchange resins or by adsorbants;
ii) adding a reagent containing iodine, alkali metal iodide and an acid to the pretreated sample, wherein the concentrations of iodine and the alkali metal iodide are adjusted in that way that essentially no color reaction with the lmw PEG but only with the hmw PEG occurs;
iii) determining optical density of the reaction mixture, eventually after a defined time;
c) for the determination of the lmw PEG concentration:
i) pretreating the sample by ion exchange resins or by adsorbants;
ii) adding a reagent containing iodine, alkali metal iodide and an acid to the pretreated sample, wherein the concentration of iodine and the alkali metal iodide is adjusted in that way that the color reaction occurs with the lmw PEG;
ii) determining optical density of the reaction mixture, eventually after a defined time.

15. The method according to claim 13, comprising the steps of:
a) collecting a sample of urine from an individual after oral administration of high molecular weight polyethylene glycol (hmw PEG) and low molecular weight polyethylene glycol (lmw PEG);
b) for the determination of the hmw PEG concentration:
i) pretreating the urine sample by ions exchange resins or adsorbent(s),
ii) adding a reagent containing iodine, alkali metal iodide and an acid to the pretreated urine sample, wherein the concentrations of iodine and the alkali metal iodide are adjusted in that way that essentially no color reaction with the lmw PEG but only with the hmw PEG occurs;
iii) determining optical density of the reaction mixture, eventually after a defined time;
c) for the determination of the lmw PEG concentration:
i) pretreating the urine sample by ion exchange resins or adsorbent(s);
ii) adding a reagent containing iodine, alkali metal iodide and an acid to the pretreated urine sample, wherein the concentration of iodine and the alkali metal iodide is adjusted in that way that the color reaction occurs with the lmw PEG;
iii) determining optical density of the reaction mixture, eventually after a defined time.

16. The method of claim 13, wherein the concentrations of hmw PEG and lmw PEG in the solution or sample are calculated from the data obtained by the optical density measurements, and then a ratio of hmw PEG concentration on lmw PEG concentration is determined as a differential index.

17. The method of claim 13, wherein the solution or sample of step a) is divided in at least two portions before or after the ion exchange resin pre-treatment step and the portions obtained are separately used in steps b) and c).

18. (canceled)

19. The method of claim 13, comprising adding a reagent containing iodine, alkali metal iodide and an acid.

20. The method of claim 13, comprising adding a reagent containing iodine, alkali metal iodide and an acid, wherein the final concentration of iodine for the measurement of hmw PEG is adjusted to 0.1-5 mM.

21. The method of claim 13, comprising adding a reagent iodine, alkali metal iodide and an acid wherein the final concentration of iodine for the measurement of lmw PEG is adjusted to higher than 5 mM.

22. (canceled)

23. (canceled)

24. The method of claim 13, further comprising measuring optical density after the addition of the reagent using a wavelength between 450 and 700 nm.

25. The method claim 13, wherein the hmw PEG used has a mean molecular weight in the range of 1,000 to 200,000.

26. The method of claim 13, wherein the lmw PEG used has a mean molecular weight below 1,000,

27. A kit for qualitative and/or semi-quantitative and/or quantitative detection of polyethylene glycol in aqueous solutions or in biological fluids, wherein high molecular weight polyethylene glycol (hmw PEG) and low molecular weight polyethylene glycol (lmw PEG) concentrations are separately measured in said aqueous solution or in said sample of a biological fluid, said kit containing:
a) a reagent for detecting high molecular weight polyethylene glycol ; and
b) a reagent for detecting low molecular weight polyethylene glycol.

28. (canceled)

29. (canceled)

30. (canceled)

31. (canceled)

32. (canceled)

33. The method according to claim 1, further comprising adding a reagent containing iodine, alkali metal iodide and an acid.

34. The method of claim 1, further comprising adding a reagent containing iodine, alkali metal iodide and an acid, wherein the final concentration of iodine for the measurement of hmw PEG is adjusted to 0.1-5 mM.

35. The method of claim 1, further comprising adding a reagent containing iodine, alkali metal iodide and an acid, wherein the final concentration of iodine for the measurement of lmw PEG is adjusted to higher than 5 mM.

36. The method of claim 1, wherein the hmw PEG used has a mean molecular weight in the range of 1,000 to 200,000.

37. The method of claim 1, wherein the hmw PEG used has a mean molecular weight in the range of 6,000 to 25,000.

38. The method of claim 1, wherein the lmw PEG used has a mean molecular weight below 1,000.

39. The method of claim 1, wherein the lmw PEG used has a mean molecular weight in the range of 300 to 500.

40. The method of claim 2, wherein the measuring of PEG is performed under conditions where essentially no barium ions and essentially no lithium ions are present.
